(19) United States
(12) Patent Application Publication
(10) Pub. No.: US 2011/0243808 A1
(43) Pub. Date: Oct. 6, 2011

(54) AUTOCLAVE

(76) Inventors: Robert Ernest Fossey, Cavan (IE); Brian Donald Collins, Buckinghamshire (GB)

(21) Appl. No.: 13/133,377

(22) PCT Filed: Dec. 11, 2009
(86) PCT No.: PCT/IE2009/000090

§ 371 Date: Jun. 7, 2011

(30) Foreign Application Priority Data
Dec. 11, 2008 (IE) S2008/0985

(51) Int. Cl. A61L 2/04

(52) U.S. Cl. 422/295; 422/307

(57) ABSTRACT

An autoclave (1) comprises a carrier framework (4) within which a drum (8) is rotatably mounted and rotated by a drive motor (12). The drum (8) defines a hollow interior region within which the waste material is treated, and an open mouth (15) at one end of the drum (8) is closed by a hingedly mounted closure member (16). The carrier framework (4) is pivotally coupled to a ground engaging mounting framework (2) by decoupleable first and second pivot mountings (5,6) at respective opposite ends (10,11) of the carrier framework (4) so that on release of the second pivot mountings (6) the carrier framework (4) and the drum (8) are pivotal about the first pivot mountings (5) by first main drive rams (19) from a horizontal operating state to an inclined loading state with the drum (8) inclined upwardly to the open mouth (15) for loading waste to be treated into the drum (8). On release of the first pivot mountings (5), the carrier framework (4) and the drum (8) are pivotal about the second pivot mountings (6) by second main drive rams (20) from the operating state to a discharge state with the drum (8) inclined downwardly towards the open mouth (15) for discharge of treated waste material from the drum (8).

The present invention relates to an autoclave, and in particular, though not limited to an autoclave for treating waste material, which may be industrial waste, domestic waste and the like.

Autoclaves for treating waste material are known. PCT Published Application Specification No. WO 2004/089547 discloses such an autoclave. In general, such autoclaves comprise a ground mounted base framework which pivotally carries a carrier framework, within which a drum for treating the waste material is carried. The drum is of circular transverse cross-section and defines a longitudinally extending central axis and a hollow interior region within which the waste material is treated. An end cap closes one end of the drum, while the other end of the drum defines an open mouth for accommodating the waste material into and out of the hollow interior region. A hingedly mounted lid closes the open mouth.

The drum is rotatably carried in the carrier framework and is rotatable about its central axis. A pivot mounting pivotally couples the carrier framework to the base framework about a pivot axis which extends transversely relative to the central axis of the drum. The carrier framework and the drum are pivotal about the pivot axis between an operating state with the drum extending substantially horizontally during which the drum is rotated during treating of the waste material therein, and a loading state with the drum tilted in a generally upwardly direction towards the open mouth for loading the drum through the open mouth. The carrier framework and the drum are also pivotal about the pivot axis from the operating state to a discharge state with the drum tilted in a generally downwardly direction towards the open mouth for discharging waste material therefrom through the open mouth.

In general, the pivot mounting about which the carrier framework and the drum are pivotal is located between the respective opposite ends of the drum, and in general, towards a mid position between the respective opposite ends of the drum. Accordingly, in order to achieve a sufficient downward tilt on the drum for discharging waste material from the drum, the pivot mounting must be set at a relatively high level above the ground. This results in two problems. Firstly, when the drum is in the operating state extending substantially horizontally, the drum must be at a relatively high level above the ground. Secondly, because of the relatively high level of the pivot axis when the drum is in the loading state, the open mouth is, in general, at an unacceptably high level above the ground.

There is therefore a need for an autoclave which addresses these problems.

The present invention is directed towards providing such an autoclave.

According to the invention there is provided an autoclave comprising a carrier means, a drum carried on the carrier means and defining a longitudinally extending central axis and a hollow interior region in which material is treated, a first pivot mounting means and a second pivot mounting means defining respective first and second pivot axes about which the carrier means is selectively and alternatively pivotal, the first and second pivot axes extending transversely relative to the central axis of the drum and being spaced apart from each other.

In one embodiment of the invention the carrier means is selectively pivotal about the first pivot axis between an operating state with the drum in an operating orientation and a loading state with the drum in a loading orientation, and the carrier means is selectively pivotal about the second pivot axis between the operating state and a discharge state with the drum in a discharge orientation.

Preferably, the first and second pivot mounting means comprise respective first and second decoupleable pivot mounting means, so that when the second pivot mounting means is decoupled, the carrier means is pivotal about the first pivot axis, and when the first pivot mounting means is decoupled, the carrier means is pivotal about the second pivot axis.

In one embodiment of the invention a ground engaging mounting means is provided, and the carrier means is releasably and pivotally coupled to the ground engaging mounting means by the first and second pivot mounting means.

In another embodiment of the invention each of the first and second pivot mounting means comprises a first coupling element located on the ground engaging mounting means and having a first pivot bore, a second coupling element located on the carrier means and having a second pivot bore, and a pivot shaft defining the corresponding one of the first and second pivot axes being engageable with the first and second pivot bores for facilitating pivoting of the first and second coupling elements relative to each other about the corresponding one of the first and second pivot axes.

In another embodiment of the invention each pivot shaft is operable between an engaged state engaging the first and second pivot bores of the corresponding ones of the first and second coupling elements, and a release state with the pivot shaft disengaged from at least one of the first and second coupling elements for releasing the carrier means from the first coupling element of the corresponding one of the first and second pivot mounting means.

Preferably, each pivot shaft is tapered, and the first and second pivot bores of the corresponding first and second coupling elements are correspondingly tapered for engaging the pivot shaft when the pivot shaft is in the engaged state.

Advantageously, an urging means is provided for urging the pivot shaft of each one of the first and second pivot mounting means between the engaged state and the release state.

Ideally, the first and second pivot mounting means are located at respective opposite ends of the carrier means relative to the central axis of the drum.

In one embodiment of the invention a pair of first pivot mounting means is provided, the first pivot mounting means defining the first pivot axis, and being spaced apart along the first pivot axis and preferably, a pair of second pivot mounting means is provided, the second pivot mounting means defining the second pivot axis, and being spaced apart along the second pivot axis.

Ideally, a drive means is provided for urging the carrier means between the operating state and the loading state and between the operating state and the discharge state. Ideally, the drive means comprises a first drive means for urging the carrier means between the operating state and the loading state, and a second drive means for urging the carrier means between the operating state and the discharge state.

Ideally, the first drive means is located at the end of the carrier means opposite to the end at which the first pivot mounting means is located, and the second drive means is located at the end of the carrier means opposite to the end at which the second pivot mounting means is located.

In another embodiment of the invention at least one blade extends into the hollow interior region of the drum, and a sparge tube is provided extending along the blade having a plurality of spaced apart jet outlets thereon for delivering a liquid into the drum, and preferably, for delivering a liquid spray into the drum.

Preferably, a plurality of circumferentially spaced apart blades are provided, and ideally, each blade is of a helical configuration, and preferably, extends substantially the length of the hollow interior region of the drum.

Ideally, the blades are equi-spaced apart circumferentially around the hollow interior region of the drum, and ideally, each blade extends into the drum from a side wall thereof.

In another embodiment of the invention the sparge tube corresponding to each blade extends along a distal edge of the blade, and in an alternative embodiment of the invention the sparge tube extends along the corresponding blade adjacent a root thereof.

In another embodiment of the invention the drum is rotatably mounted in the carrier about its longitudinal central axis, and preferably, the drum is of circular transverse cross-section.

The invention will be more clearly understood from the following description of some preferred embodiments thereof, which are given by way of example only, with reference to the accompanying drawings, in which:

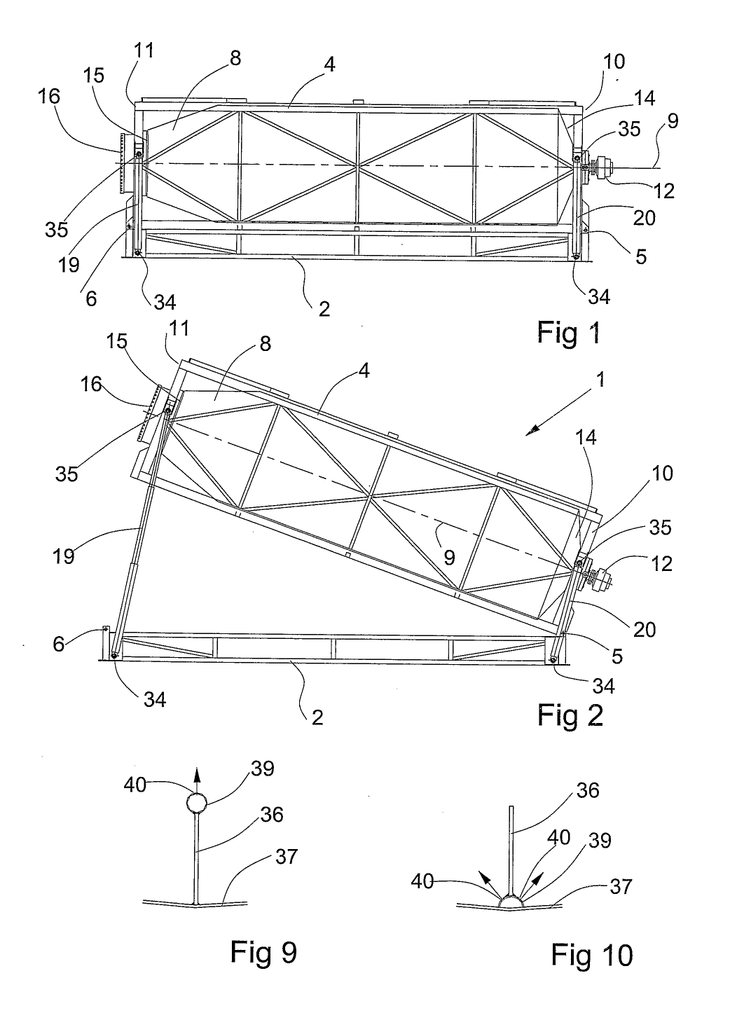

FIG. 1 is a side elevational view of an autoclave according to the invention in one orientation,

FIG. 2 is a side elevational view similar to that of FIG. 1 of the autoclave of FIG. 1 illustrating the autoclave in a different orientation to that of FIG. 1,

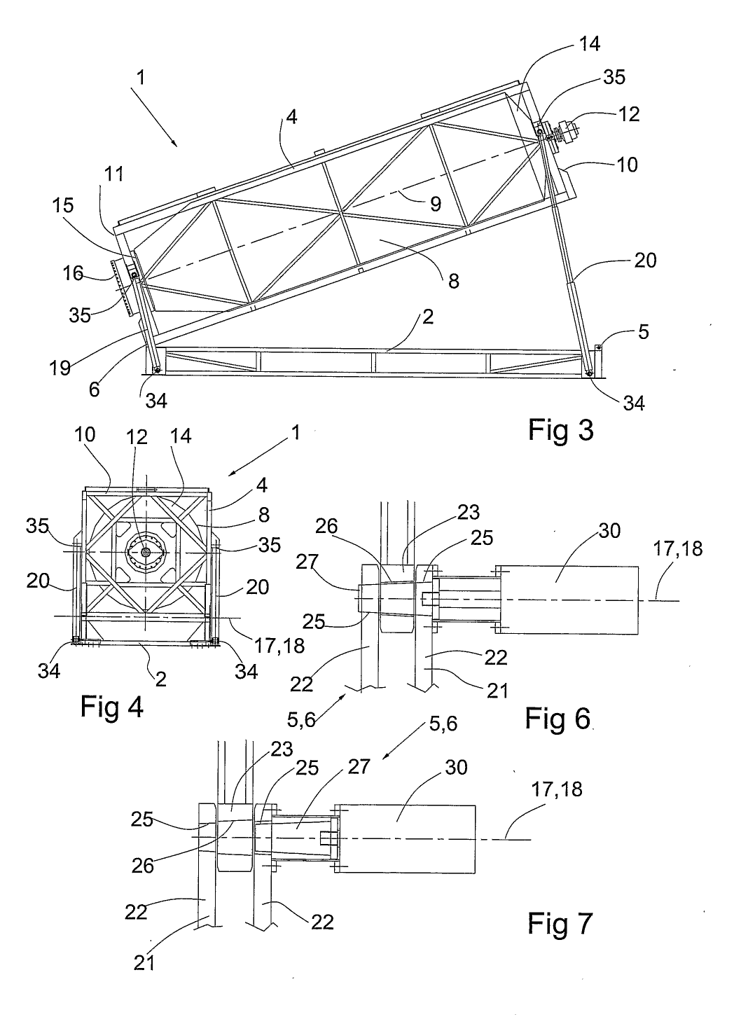

FIG. 3 is a side elevational view similar to that of FIG. 1 of the autoclave of FIG. 1 in a further different orientation to that of FIGS. 1 and 2,

FIG. 4 is an end elevational view of the autoclave of FIG. 1 in the orientation of FIG. 1,

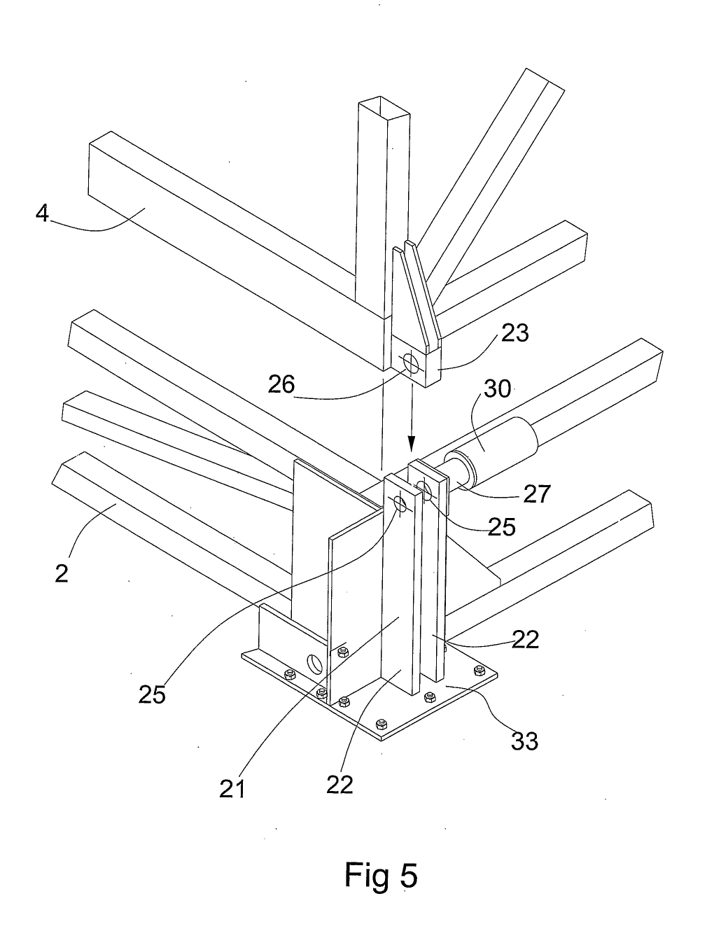

FIG. 5 is an exploded perspective view of a detail of the autoclave of FIG. 1,

FIG. 6 is a transverse cross-sectional end elevational view of a portion of the detail of FIG. 5 of the autoclave of FIG. 1,

FIG. 7 is a view similar to that of FIG. 6 of the detail of FIG. 6 illustrating a portion of the detail in a different state to that of FIG. 6,

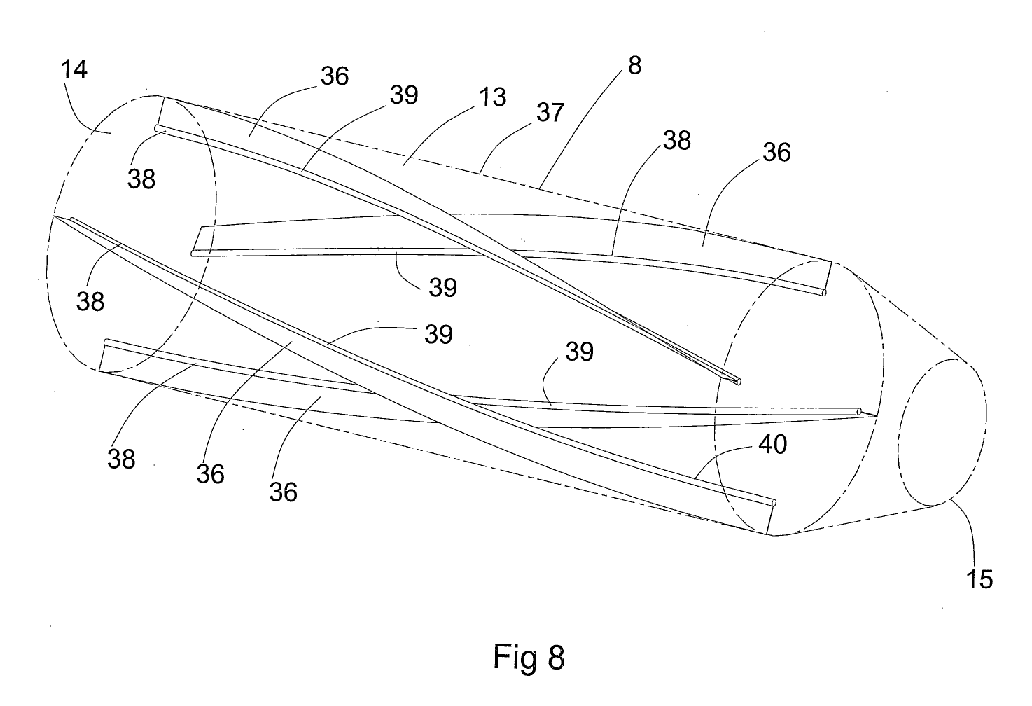

FIG. 8 is a diagrammatic perspective view of another portion of the autoclave of FIG. 1,

FIG. 9 is an end elevational view of a detail of the portion of the autoclave of FIG. 8, and

FIG. 10 is an end elevational view similar to FIG. 9 of a portion similar to that of FIG. 8 of an autoclave according to another embodiment of the invention.

Referring to the drawings and initially to FIGS. 1 to 9, there is illustrated an autoclave according to the invention, indicated generally by the reference numeral 1 for treating waste material, which may be industrial, domestic or any other suitable treatable waste, which in general, is treated under hot, humid conditions. The operation of such autoclaves 1 will be well known to those skilled in the art, and is described in PCT Published Application Specification No. WO 2004/089547. The autoclave 1 comprises a ground engaging mounting means, namely, ground engaging mounting framework 2 which is adapted to be mounted on the ground, and which pivotally carries a carrier means, in this embodiment of the invention a carrier framework 4 on first and second pivot mounting means, namely, first and second pivot mountings 5 and 6, respectively, as will be described below.

A drum 8 is rotatably mounted in the carrier framework 4 and is rotatable therein about a longitudinally extending central rotational axis 9. Bearings (not shown) mounted in respective opposite ends 10 and 11 of the carrier framework 4 rotatably carry the drum 8 about the central rotational axis 9. A drive motor 12 mounted on the carrier framework 4 at the end 10 rotates the drum 8 about the central rotational axis 9. The drum 8 is of circular transverse cross-sectional area and defines the central rotational axis 9, as well as defining a hollow interior region 13, see FIG. 8, within which the waste material is treated. An end cap 14 closes one end of the drum 8 adjacent the end 10 of the carrier framework 4, and the other end of the drum 8 defines an open mouth 15, which is sealably closed by a hingedly mounted closure member 16 adjacent the end 11 of the carrier framework 4. This aspect of the drum 8 is described in PCT Published Application Specification No. WO 2004/089547.

The first and second pivot mountings 5 and 6 are decoupleable pivot mountings for releasably coupling the carrier framework 4 to the ground engaging mounting framework 2, and define respective first and second pivot axes 17 and 18, respectively, which extend transversely of the central rotational axis 9 of the drum 8, so that the carrier framework 4 is selectively and alternately pivotal about the respective first and second pivot axes 17 and 18. Thus, with the carrier framework 4 released from the ground engaging mounting framework 2 by the second pivot mounting 6, the carrier framework 4 is pivotal about the first pivot axis 17 between an operating state illustrated in FIG. 1 with the drum 8 extending horizontally in an operating orientation, and an upwardly inclined loading state illustrated in FIG. 2 with the drum 8 inclined upwardly towards the open mouth 15 in a loading orientation for loading thereof through the open mouth 5. With the carrier framework 4 released from the ground engaging mounting framework 2 by the first pivot mounting 5, the carrier framework 4 is pivotal about the second pivot axis 18 between the operating state and a discharge state illustrated in FIG. 3 with the drum 8 inclined downwardly towards the open mouth 15 in a discharge orientation for discharging material from the hollow interior region 13 of the drum 8 through the open mouth 15.

A drive means comprising first and second drive means provided by two pairs of first main drive rams 19 and two pairs of second main drive rams 20, respectively, act between the ground engaging mounting framework 2 and the carrier framework 4 for selectively and alternately pivoting the carrier framework 4 between the operating state and the loading state and between the operating state and the discharge state, respectively, as will be described in more detail below.

Turning now in more detail to the first and second pivot mountings 5 and 6, the first and second pivot mountings 5 and 6 are located at respective opposite ends of the ground engaging mounting framework 2, and two first pivot mountings 5 and two second pivot mountings 6 are provided at respective opposite corners of the ground engaging mounting framework 2. Each first and second pivot mounting 5 comprises a first coupling element 21 comprising a pair of spaced apart mounting plate members 22 mounted on the ground engaging mounting framework 2, and a second coupling element 23 mounted on the carrier framework 4. A first pivot bore 25 extends through each mounting plate member 22, and a second pivot bore 26 extends through each second coupling element 23 for pivotally engaging a corresponding pivot shaft 27. The pivot shafts 27 of the respective first pivot mountings 5 define the first pivot axis 17, and the pivot shafts 27 of the respective second pivot mountings 6 define the second pivot axis 18.

Each pivot shaft 27 is urgeable by an urging means, namely, a corresponding secondary ram 30 mounted on the ground engaging mounting framework 2 between an engaged state illustrated in FIG. 6 with the pivot shaft 27 engaging the first and second pivot bores 25 and 26 of the corresponding one of the first and second coupling elements 21 and 23, and a release state illustrated in FIG. 7 with the pivot shaft 27 disengaged from the second pivot bore 26 of the second coupling element 23 and the first pivot bore 25 of one of the mounting plate members 22 for releasing the carrier framework 4 from the ground engaging mounting framework 2 adjacent the corresponding one of the first and second pivot mountings 5 and 6. The pivot shafts 27 are tapered towards their respective distal ends, and the first and second pivot bores 25 and 26 in the mounting plate members 22 and the second coupling element 23 are correspondingly tapered for engaging the corresponding pivot shaft 27 when the pivot shaft 27 is in the engaged state. Tapering of the pivot shafts 27 and the first and second pivot bores 25 and 26 facilitates ease of engagement of the pivot shafts 27 with the corresponding first and second pivot bores 25 and 26.

The first plate members 22 of the first coupling element 21 are secured by welding to the ground engaging mounting framework 2 and extend downwardly to terminate in corresponding ground engaging plates 33 which are provided for securing the ground engaging mounting framework 2 to the ground by suitable fixings, for example, masonry bolts. The second coupling elements 23 are welded to the carrier framework 4 at the respective opposite ends 10 and 11 thereof.

Returning now to the first and second main drive rams 19 and 20, in order to provide sufficient slewing of the carrier framework 4 and in turn the drum 8 about the first and second pivot axes 17 and 18, the first and second main drive rams 19 and 20 are provided in a plurality of telescoping sections. Pivotal couplings 34 and 35 pivotally couple the first and second main drive rams 19 and 20 to the ground engaging mounting framework 2 and the carrier framework 4, respectively.

Referring now in particular to FIGS. 8 and 9, four circumferentially equi-spaced apart blades 36 of helical configuration extend from a side wall 37 inwardly into the hollow interior region 13 of the drum 8 for tumbling and agitating waste material within the hollow interior region 13 of the drum 8 as the drum 8 rotates. Each blade 36 extends substantially the length of the drum 8 and terminates at its longitudinally extending distal edge 38 in a sparge tube 39 which extends along the length of the distal edge of the blade 36. A plurality of longitudinally spaced apart jet outlets 40 are provided along the length of each sparge tube 39 for delivering jets of water in the form of a spray into the hollow interior region 13 for maintaining the moisture content of the waste material in the drum 8 during processing thereof at a desired level. Suitable connections are provided for delivering water to the sparge tubes 39 as the drum 8 rotates. A typical arrangement for delivering water into a rotating drum, which would be suitable for delivering water to the sparge tube 39 as the drum 8 rotates is described in PCT Published Application Specification No. WO 2004/089547.

In use, during normal operation of the autoclave 1 in the processing of the waste material, the carrier framework 4 is secured to the ground engaging mounting framework 2 by the first and second pivot mountings 5 and 6 by virtue of the respective pivot shafts 27 being in the engaged state engaging the first and second pivot bores 25 and 26 in the first and second coupling elements 21 and 23. With the pivot shafts 27 in the engaged state, the carrier framework 4 is rigidly secured to the ground engaging mounting framework 2, and the first and second main drive rams 19 and 20 are in the retracted state with the carrier framework 4 and the drum 8 in the operating state and with the central rotational axis 9 of the drum 8 extending horizontally.

To load the drum 8 with waste material, the pivot shafts 27 of the second pivot mountings 6 are urged into the release state for decoupling the corresponding second coupling elements 23 from the corresponding first coupling elements 21, for in turn releasing the carrier framework 4 from the ground engaging mounting framework 2 adjacent the second pivot mountings 6. With the pivot shafts 27 of the first pivot mountings 5 in the engaged state pivotally coupling the first and second coupling elements 21 and 23 thereof, the first main drive rams 19 are operated for pivoting the carrier framework 4 about the first pivot axis 17 into the loading state with the carrier framework 4 and the drum 8 tilted in an upwardly inclined orientation towards the open mouth 15 of the drum 8. The closure member 16 is operated into the open state, and waste material is loaded into the hollow interior region 13 of the drum 8 through the open mouth 15. On completion of loading of the drum 8, the closure member 16 is operated to sealably close the open mouth 15, and the first main drive rams 19 are operated for pivoting the carrier framework 4 about the first pivot axis 17 into the operating state.

In the operating state the pivot shafts 27 of the second pivot mountings 6 are operated into the engaged state for coupling the corresponding first and second coupling elements 21 and 22, and in turn for coupling the carrier framework 4 to the ground engaging mounting framework 2 adjacent the second pivot mountings 8. With the pivot shafts 27 of the first and second pivot mountings 5 and 6 operated into the engaged state for securing the carrier framework 4 to the ground engaging mounting framework 2, the drum 8 is rotated by the drive motor 12, and the waste material in the drum 8 is brought up to the appropriate processing temperature by a suitable heat exchange medium, such as heated oil, which is circulated in passageways extending longitudinally along the outer surface of the drum 8 as described in PCT Published Application Specification No. WO 2004/089547. The moisture content of the waste material in the drum 8 is maintained constant by delivering a water spray into the hollow interior region 13 of the drum 8 through the sparge tubes 39. The water may be hot or cold, but in general will be heated.

To discharge treated waste material from the drum 8, the pivot shafts 27 of the first pivot mountings 5 are operated into the release state for decoupling the corresponding second coupling elements 23 from the corresponding first coupling elements 21, and in turn for decoupling the carrier framework 4 from the ground engaging mounting framework 3 adjacent the first pivot mountings 5, to thereby facilitate pivoting of the carrier framework 4 about the second pivot axis 18. The second main drive rams 20 are operated for urging the carrier framework 4, and in turn the drum 8 into the discharge state with the carrier framework 4 and the drum 8 inclined downwardly towards the open mouth 15 of the drum 8. The closure member 16 is again urged into the open state and the treated waste material is discharged from the drum 8 through the open mouth 15. On completion of discharge of the waste material, the second main drive rams 20 are operated for urging the carrier framework 4 and the drum 8 into the operating state. The pivot shafts 27 of the second pivot mountings 6 are urged into the engaged state for again coupling the first and second coupling elements 21 and 23 of the corresponding second pivot mountings 6.

Referring now to FIG. 10, there is illustrated a blade 36 with a corresponding sparge tube 39 of an autoclave (not shown) according to another embodiment of the invention. In this embodiment of the invention the sparge tube 39 is located at the proximal end or root of the corresponding blade 36 and extends the length of the blade 36. Longitudinally spaced apart jet outlets 40 are located along the length of the sparge tube 39 on respective opposite sides of the corresponding blade 36 for delivering water into the hollow interior region 13 of the drum 8. Otherwise, the autoclave according to this embodiment of the invention and its operation is similar to that already described with reference to the autoclave 1 of FIGS. 1 to 9.

The advantages of the invention are many. A particularly important advantage of the invention is that the carrier framework 4, and in turn the drum 8 can be mounted at a much lower level than drums of autoclaves known heretofore. By virtue of the fact that the carrier framework is selectively pivotal at respective opposite ends of the ground engaging mounting framework 2, the level of the drum 8 when in the operating orientation is significantly lower than the level of the drum in the operating state of autoclaves known heretofore. Additionally, when the carrier framework 4 and the drum 8 are pivoted into the loading state, the level of the open mouth 15 of the drum 8 in the loading orientation is significantly lower than in autoclaves known heretofore. In fact, the first and second pivot mountings 5 and 6 can be located as close to the ground as desired, and in general, can be located at a level so that when the carrier framework 4 is in the discharge state with the drum 8 in the discharge orientation, a take-off conveyor belt for carrying off the treated waste material can be located just below the open mouth.

While the autoclave according to the invention has been described as comprising a pair of first pivot mountings and a pair of second pivot mountings, in certain cases, it is envisaged that a single first pivot mounting and a single second pivot mounting may be sufficient. It is also envisaged that while the first and second pivot mountings have been described as being located at the respective opposite ends of the ground engaging mounting framework and at the appropriate ends of the carrier framework, while this is preferable, in certain cases, it is envisaged that the first and second pivot mountings may be located somewhat inwardly relative to the respective opposite ends of the drum.

It will also be appreciated that while specific constructions of first and second pivot mountings have been described, any other suitable pivot mounting means may be provided. It will also be appreciated that other suitable urging means besides hydraulic rams may be provided for urging the pivot shafts of the respective first and second pivot mountings between the engaged and disengaged states, and it is also envisaged that other suitable main drive means besides drive rams may be used for pivoting the carrier framework and the drum between the operating and loading states and between the operating and discharge states.

Additionally, while the autoclave has been described as comprising a ground engaging mounting framework and a carrier framework of specific constructions, any other suitable ground engaging mounting means and any other suitable carrier means may be provided. Indeed, it is envisaged that in certain cases the first and second pivot mountings may be mounted directly on the ground.

While the drum has been described as comprising four circumferentially equi-spaced apart blades, it will be appreciated that any number of blades may be provided from one upwards, and while it is preferable, it is not essential that the blades be equi-spaced. Additionally, it is not essential that each blade be provided with a sparge tube.

1-26. (canceled)

27. An autoclave comprising a carrier means, a drum carried on the carrier means and defining a longitudinally extending central axis and a hollow interior region in which material is treated, a first pivot mounting means and a second pivot mounting means defining respective first and second pivot axes about which the carrier means is selectively and alternatively pivotal, the first and second pivot axes extending transversely relative to the central axis of the drum and being spaced apart from each other.

28. An autoclave as claimed in claim 27 in which the carrier means is selectively pivotal about the first pivot axis between an operating state with the drum in an operating orientation and a loading state with the drum in a loading orientation, and the carrier means is selectively pivotal about the second pivot axis between the operating state and a discharge state with the drum in a discharge orientation.

29. An autoclave as claimed in claim 27 in which the first and second pivot mounting means comprise respective first and second decoupleable pivot mounting means, so that when the second pivot mounting means is decoupled, the carrier means is pivotal about the first pivot axis, and when the first pivot mounting means is decoupled, the carrier means is pivotal about the second pivot axis.

30. An autoclave as claimed in claim 27 in which a ground engaging mounting means is provided, and the carrier means is releasably and pivotally coupled to the ground engaging mounting means by the first and second pivot mounting means.

31. An autoclave as claimed in claim 30 in which each of the first and second pivot mounting means comprises a first coupling element located on the ground engaging mounting means and having a first pivot bore, a second coupling element located on the carrier means and having a second pivot bore, and a pivot shaft defining the corresponding one of the first and second pivot axes being engageable with the first and second pivot bores for facilitating pivoting of the first and second coupling elements relative to each other about the corresponding one of the first and second pivot axes.

32. An autoclave as claimed in claim 31 in which each pivot shaft is operable between an engaged state engaging the first and second pivot bores of the corresponding ones of the first and second coupling elements, and a release state with the pivot shaft disengaged from at least one of the first and second coupling elements for releasing the carrier means from the first coupling element of the corresponding one of the first and second pivot mounting means.

33. An autoclave as claimed in claim 31 in which each pivot shaft is tapered, and the first and second pivot bores of the corresponding first and second coupling elements are correspondingly tapered for engaging the pivot shaft when the pivot shaft is in the engaged state.

34. An autoclave as claimed in claim 32 in which an urging means is provided for urging the pivot shaft of each one of the first and second pivot mounting means between the engaged state and the release state.

35. An autoclave as claimed in claim 27 in which the first and second pivot mounting means are located at respective opposite ends of the carrier means relative to the central axis of the drum.

36. An autoclave as claimed in claim 27 in which a pair of first pivot mounting means is provided, the first pivot mounting means defining the first pivot axis, and being spaced apart along the first pivot axis, ad preferably, a pair of second pivot mounting means is provided, the second pivot mounting means defining the second pivot axis, and being spaced apart along the second pivot axis.

37. An autoclave as claimed in claim 27 in which a drive means is provided for urging the carrier means between the operating state and the loading state and between the operating state and the discharge state.

38. An autoclave as claimed in claim 37 in which the drive means comprises a first drive means for urging the carrier means between the operating state and the loading state, and a second drive means for urging the carrier means between the operating state and the discharge state.

39. An autoclave as claimed in claim 38 in which the first and second drive means are located at respective opposite ends of the carrier means relative to the central axis of the drum, and preferably, the first drive means is located at the end of the carrier means opposite to the end at which the first pivot mounting means is located, and the second drive means is located at the end of the carrier means opposite to the end at which the second pivot mounting means is located.

40. An autoclave as claimed in claim 27 in which the drum is rotatably mounted in the carrier means about its longitudinal central axis, and preferably, the drum is of circular transverse cross-section.

41. An autoclave as claimed in claim 27 in which at least one blade extends into the hollow interior region of the drum, and a sparge tube is provided extending along the blade having a plurality of spaced apart jet outlets thereon for delivering a liquid into the drum.

42. An autoclave as claimed in claim 41 in which the jet outlets of the sparge tube are adapted for delivering a liquid spray into the drum.

43. An autoclave as claimed in claim 41 in which a plurality of circumferentially spaced apart blades are provided.

44. An autoclave as claimed in claim 41 in which each blade is of a helical configuration.

45. An autoclave as claimed in claim 41 in which each blade extends substantially the length of the hollow interior region of the drum, and preferably, the blades are equi-spaced apart circumferentially around the hollow interior region of the drum.

46. An autoclave as claimed in claim 41 in which each blade extends into the drum from a side wall thereof, and preferably, the sparge tube corresponding to each blade extends along a distal edge of the blade, and advantageously, the sparge tube corresponding to each blade extends along a root of the blade thereof.
